(19) United States
(12) Patent Application Publication
(10) Pub. No.: US 2017/0157041 A1
(43) Pub. Date: Jun. 8, 2017

(54) CANNABIS TABLET OR CAPSULE

(71) Applicant: Stephen Goldner, West Bloomfield, MI (US)

(72) Inventor: Stephen Goldner, West Bloomfield, MI (US)

(21) Appl. No.: 15/369,158

(22) Filed: Dec. 5, 2016

Related U.S. Application Data
(60) Provisional application No. 62/262,957, filed on Dec. 4, 2015.

(51) Int. Cl. A61K 9/00; A61K 9/20; B33Y 50/02; B29C 67/00; B33Y 30/00; B33Y 70/00; A61K 31/352; B33Y 80/00

(52) U.S. Cl. CPC A61K 9/0056; A61K 31/352; A61K 9/2059; A61K 9/2018; A61K 9/2054; B33Y 80/00; B29C 67/0088; B29C 67/0055; B33Y 30/00; B33Y 70/00; B33Y 50/02; B29K 2001/00

(57) ABSTRACT

An orally dissolvable cannabis tablet includes one or more active cannabis-based chemical constituents and one or more inactive constituents. The active cannabis-based chemical constituents includes at least one of cannabinoids or terpenoids.

### REFERENCE TO RELATED APPLICATIONS

This application claims priority to U.S. Provisional Application No. 62/262,957 filed on Dec. 4, 2015.

### BACKGROUND

Among other purposes, cannabis is used for medicinal reasons to treat disease or alleviate symptoms. For example, the active chemicals in medicinal cannabis can be delivered into the body through inhalation, ingestion, or topical application.

### BRIEF DESCRIPTION OF THE DRAWINGS

FIG. 1 illustrates an example orally dissolvable cannabis tablet.

FIG. 2

FIG. 3 illustrates an example system or machine that is configured to additively form an orally dissolvable cannabis tablet.

### DESCRIPTION

The amount of time for the active chemical of cannabis to take effect varies according to the delivery method, with inhalation being the most rapid. Although inhalation is the quickest, there are well known detrimental effects to smoking that make inhalation less attractive for medical uses. In this regard, as further discussed below, an orally dissolvable cannabis tablet can be used to rapidly deliver the active chemicals of cannabis without many of the detrimental effects associated with smoking.

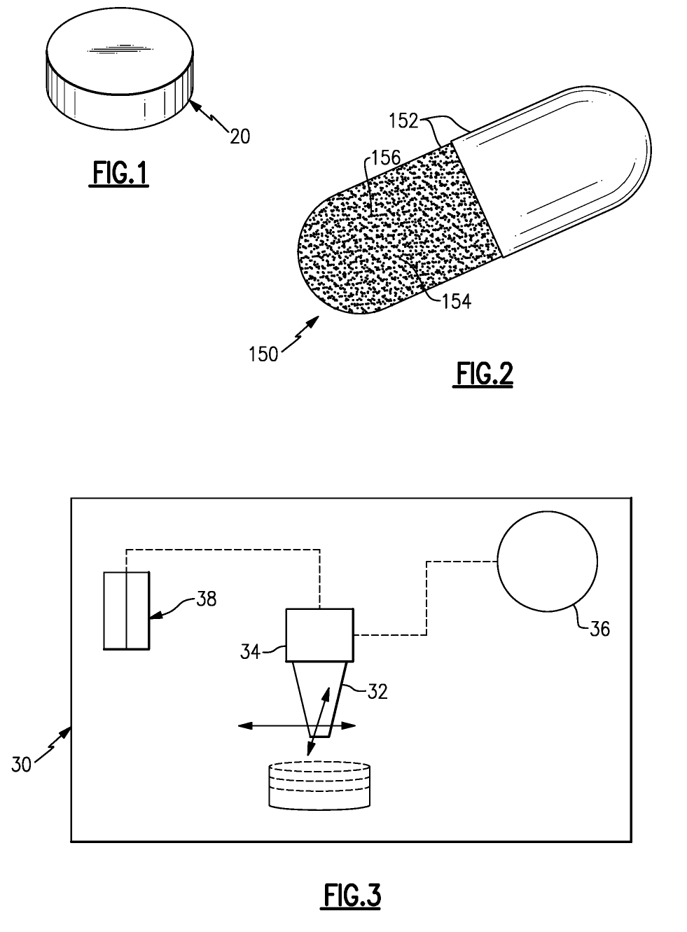

FIG. 1 shows an example orally dissolvable cannabis tablet 20. Although depicted with a generally cylindrical geometry, it is to be understood that the tablet can alternatively be provided in other geometries that are suitable for oral use and in any of a variety of sizes. Typical tablet sizes may include, but are not limited to, tablets of total weight less than 100 milligrams, tablets of total weight of 100-200 milligrams, tablets of total weight of 200-500 milligrams, or tablets of total weight greater than 500 milligrams or greater than 1000 milligrams.

The orally dissolvable cannabis tablet 20 includes one or more active cannabis-based chemical constituents and one or more inactive constituents. The one or more active cannabis-based chemical constituents are chemical constituents that are derived from cannabis or synthetic constituents that are chemically similar in structure and effect to constituents from natural cannabis. Generally, the active cannabis-based chemical constituents can include one or more constituents selected from several chemical classes, including cannabinoids and terpenoids. For example, the cannabinoids can include, but are not limited to, tetrahydrocannabinol (delta-g-tetrahydrocannabinol, commonly known as “THC”), cannabidiol, cannabinol, cannabavarin, cannabigerol, cannabichromene, delta-8-THC, cannabicyclol, cannabitriol, and cannabielsoin. The terpenoids can include, but are not limited to, myrcene, limonene, and caryophyllene. These and other active cannabis-based chemical constituents can be extracted from cannabis for use in the orally dissolvable cannabis tablet 20 using known extraction techniques.

The one or more inactive constituents, or excipients, in the orally dissolvable cannabis tablet 20 are chemical constituents that provide dissolvability and, optionally, other functionalities. For example, the predominant inactive constituent by weight can include a water-soluble solid. Examples of water-soluble solids can include, but are not limited to, starches, sugars, and cellulose solids that readily dissolve in water such that upon insertion of the orally dissolvable cannabis tablet 20 into the mouth, the orally dissolvable cannabis tablet 20 dissolves in a relatively short period of time. As a result of the dissolution, the one or more active cannabis-based chemical constituents are rapidly absorbed through the walls of the mouth, such as under the tongue, through the gums, or through the cheek, into the bloodstream for near immediate effect.

In further examples, the one or more inactive constituents also include one or more processing aids. The processing aids are constituents that provide functionality for fabricating the orally dissolvable cannabis tablet 20. For example, the processing aids can include, but are not limited to, binders and release agents. For example, a binder may be used to hold all of the constituents of the orally dissolvable cannabis tablet 20 together in the desired tablet geometry, and a release agent may be used to facilitate release from a die or supporting substrate during fabrication.

In further examples, the one or more inactive constituents also include one or more enhancers. The enhancers are constituents that modify the chemical stability (e.g., preservatives), flavor, color, fragrance, or the like, of the orally dissolvable cannabis tablet 20. One example modifier includes caffeine. In further examples, the excipients may include one or more constituents from Table 1 herein.

In further examples, the composition of the orally dissolvable cannabis tablet 20 includes 1-50 milligrams or 1-100 milligrams of the one or more active cannabis-based chemical constituents and a remainder of the inactive constituents. Most typically, the orally dissolvable cannabis tablet 20 includes 2-30 milligrams of the one or more active cannabis-based chemical constituents and a remainder of the inactive constituents.

FIG. 2 illustrates an example of a capsule 150. In this example, the capsule 150 includes an outer shell 152 that contains one or more active cannabis-based chemical constituents 154 therein. The shell 152 may also contain one or more inactive constituents, or excipients 156, which may be mixed with or separate from the active cannabis-based chemical constituents 154. The cannabis-based chemical constituents 154, the excipients 156, or both may be in solid, powdered form. Alternatively, the cannabis-based chemical constituents 154 and the excipients 156 may be in liquid form. The shell 152 may be a multi-piece shell or a single-piece shell. Although not limited, the shell 152 most typically is formed of gelatin.

In one example, the capsule 150 has a total size of 15-400 milligrams, with 1-150 or 1-250 milligrams of the active cannabis-based chemical constituents 154, and a remainder of inactive constituents or excipients. For instance, the active cannabis-based chemical constituents 154 in the capsule 150 includes 1-100 milligrams of THC and 1-50 milligrams of CBD. In another example, the active cannabis-based chemical constituents 154 in the capsule 150 includes a combined amount of 1-150 milligrams of cannabinol and at least one of THC and CBD. In one example, the combined amount includes 1-50 of cannabinol.

In further examples, the one or more active cannabis-based chemical constituents of the capsule 150 are chemical constituents that are derived from cannabis or synthetic constituents that are chemically similar in structure and effect to constituents from natural cannabis. Generally, the active cannabis-based chemical constituents can include one or more constituents selected from several chemical classes, including cannabinoids and terpenoids. For example, the cannabinoids can include, but are not limited to, tetrahydrocannabinol (delta-g-tetrahydrocannabinol, commonly known as “THC”), cannabidiol, cannabinol, cannabavarin, cannabigerol, cannabichromene, delta-8-THC, cannabicyclol, cannabitriol, and cannabielsoin. The terpenoids can include, but are not limited to, myrcene, limonene, and caryophyllene. These and other active cannabis-based chemical constituents can be extracted from cannabis for use in the capsule 150 using known extraction techniques.

The one or more inactive constituents, or excipients, in the capsule 150 are chemical constituents that provide dissolvability and, optionally, other functionalities. For example, the predominant inactive constituent by weight can include a water-soluble solid. Examples of water-soluble solids can include, but are not limited to, starches, sugars, and cellulose solids that readily dissolve in water such that upon ingestion, the capsule 150 dissolves and absorbs into the body in a relatively short period of time.

In further examples, the one or more inactive constituents also include one or more processing aids. The processing aids are constituents that provide functionality for fabricating the capsule 150. For example, the processing aids can include, but are not limited to, binders and release agents.

In further examples, the one or more inactive constituents also include one or more enhancers. The enhancers are constituents that modify the chemical stability (e.g., preservatives), flavor, color, fragrance, or the like, of the capsule 150. One example modifier includes caffeine. In further examples, the excipients may include one or more constituents from Table 1 below.

TABLE 1
Excipients

| Excipient | Use |
|---|---|
| Acacia | Emulsifying agent; |
| | stabilizing agent; |
| | suspending agent; |
| | tablet binder; |
| | viscosity-increasing |
| | agent |
| Alginate | Binder |
| Alginic Acid | Stabilizing agent; |
| | suspending agent; |
| | tablet binder, |
| | tablet disintegrant; |
| | viscosity-increasing |
| | agent. |
| Aluminum Acetate | Antiseptic |
| Benzyl Alcohol | Antimicrobial |
| | preservative; |
| | disinfectant; solvent |
| Butyl Paraben | Antimicrobial |
| | preservative |
| Butylated Hydroxy | Antioxidant. |
| Toluene | |
| Citric acid | Disintegrant |
| Calcium carbonate | Tablet and capsule |
| | diluent; therapeutic |
| | agent |
| Candelilla wax | Binder |
| Croscarmellose sodium | Tablet and capsule |
| | disintegrant |
| Confectioner sugar | Sugar coating adjunct; |
| | sweetening agent; |
| | tablet and capsule |
| | diluents |
| Colloidal silicone | Adsorbent; anticaking |
| dioxide | agent; emulsion |
| | stabilizer; glidant; |
| | suspending agent; tablet |
| | disintegrant; thermal |
| | stabilizer; |
| | viscosity-increasing |
| | agent. |
| Cellulose | Adsorbent; suspending |
| | agent; tablet and |
| | capsule diluent; |
| | tablet |
| | disintegrant.(cellulose |
| | microcrystaline) |
| | Adsorbent; glidant; |
| | suspending agent; |
| | tablet and capsule |
| | diluent; tablet |
| | disintegrant (cellulose |
| | powdered) Tablet and |
| | capsule |
| | diluent.(cellulose |
| | Silicified) |
| Plain or anhydrous | Diluent |
| calcium phosphate | |
| Carnuba wax | Binder |
| Corn starch | Binder |
| Carboxymethylcellulose | Stabilizing agent; |
| calcium | suspending agent; |
| | tablet and capsule |
| | disintegrant; |
| | viscosity-increasing |
| | agent; water- |
| | absorbing agent |
| Calcium stearate | Tablet and capsule |
| | lubricant |
| Calcium disodium | Chelation |
| EDTA | |
| Copolyvidone | Film-former; granulating |
| | agent; tablet binder |
| Castor oil hydrogenated | Extended release agent; |
| | stiffening agent; |
| | tablet and |
| | capsule lubricant |
| Calcium hydrogen | Diluent |
| phosphate dihydrate | |
| Cetylpyridine chloride | Antimicrobial |
| | preservative; |
| | antiseptic; |
| | cationic surfactant; |
| | disinfectant; |
| | solubilizing agent; |
| | wetting agent |
| Cysteine HCL | Reducing Agent |
| Crosspovidone | Tablet disintegrant. |
| calcium phosphate di or | Tablet and capsule |
| tri basic | diluent Anticaking |
| | agent; buffer, |
| | nutrient; dietary |
| | supplement; |
| | glidant; tablet |
| | and capsule diluent and |
| | clouding agent(for |
| | calium phosphage |
| | tribasic) |
| Dibasic Calcium | Diluent |
| Phosphate | |
| Disodium hydrogen | Buffering agent |
| phosphate | |
| Dimethicone | Antifoaming agent; |
| | emollient |
| Erythrosine Sodium | Color |
| Ethyl Cellulose | Coating agent; |
| | flavoring fixative; |
| | tablet binder; |
| | tablet filler; |
| | viscosity-increasing |
| | agent. |
| Gelatin | Coating agent; |
| | film-former; |
| | gelling agent; |
| | suspending agent; |
| | tablet binder; |
| | viscosity-increasing |
| | agent |
| Glyceryl monooleate | Nonionic surfactant |
| Glycerin | Antimicrobial |
| | preservative; |
| | emollient; |
| | humectant; |
| | plasticizer; |
| | solvent; |
| | sweetening agent; |
| | tonicity agent |
| Glycine | Tonicity |
| Glyceryl monostearate | Emollient; emulsifying |
| | agent; |
| | solubilizing agent; |
| | stabilizing agent; |
| | sustained-release |
| | ingredient; |
| | tablet and capsule |
| | lubricant |
| Glyceryl behenate | Coating agent; tablet |
| | binder; tablet and |
| | capsule lubricant |
| Hydroxy propyl | Coating agent; |
| cellulose | emulsifying agent; |
| | stabilizing agent; |
| | suspending agent; |
| | tablet binder; thickening |
| | agent; |
| | viscosity-increasing |
| | agent. |
| Hydroxyl propyl methyl | Coating agent; film- |
| cellulose | former; |
| | rate-controlling polymer |
| | for sustained release; |
| | stabilizing agent; |
| | suspending agent; |
| | tablet binder; |
| | viscosity-increasing |
| | agent. |
| Hypromellose | Coating agent; film- |
| | former; |
| | rate-controlling polymer |
| | for sustained release; |
| | stabilizing agent; |
| | suspending agent; tablet |
| | binder; viscosity- |
| | increasing agent. |
| HPMC Pthalate | Coating agent. |
| Iron oxides or ferric | Color |
| oxide | |
| Iron oxide yellow | Color |
| Iron oxide red or ferric | Color |
| oxide | |
| Lactose hydrous or | Binding agent; diluent |
| anhydrous or | for dry-powder |
| monohydrate or | inhalers; |
| spray dried | lyophilization aid; |
| | tablet binder; tablet and |
| | capsule diluent. |
| | (lactose anhydrous) |
| | Binding agent; |
| | diluent for dry- |
| | powder inhalers; |
| | tablet binder; |
| | tablet and capsule |
| | diluent(lactose |
| | monhydrate) Binding |
| | agent; diluent for dry- |
| | powder inhalations; |
| | tablet and capsule |
| | diluent; |
| | tablet and capsule |
| | filler.(lactose spray |
| | dried) |
| Magnesium stearate | Tablet and capsule |
| | lubricant |
| Microcrystalline | Adsorbent; suspending |
| cellulose | agent; tablet and |
| | capsule diluent; tablet |
| | disintegrant same as |
| | cellulose see above it |
| | is just that mcc is usp |
| Mannitol | Sweetening agent; tablet |
| | and capsule diluent; |
| | tonicity agent; |
| | vehicle (bulking agent) |
| | for lyophilized |
| | preparations |
| Methyl cellulose | Coating agent; |
| | emulsifying agent; |
| | suspending agent; |
| | tablet and capsule |
| | disintegrant; tablet |
| | binder; viscosity- |
| | increasing agent |
| Magnesium carbonate | Tablet and capsule |
| | diluent |
| Mineral oil | Emollient; lubricant; |
| | oleaginous vehicle; |
| | solvent |
| Methacrylic acid | Coating |
| copolymer | |
| Magnesium oxide | Tablet and capsule |
| | diluent |
| Methyl paraben | Antimicrobial |
| | preservative |
| Povidone or PVP | Disintegrant; dissolution |
| | aid; suspending |
| | agent; tablet binder. |
| PEG | Ointment base; |
| | plasticizer; solvent; |
| | suppository base; |
| | tablet and capsule |
| | lubricant |
| Polysorbate 80 | solubilizer |
| Propylene glycol | Antimicrobial |
| | preservative; |
| | disinfectant; |
| | humectant; |
| | plasticizer; |
| | solvent; stabilizer for |
| | vitamins; water- |
| | miscible cosolvent. |
| Polyethylene oxide | Mucoadhesive; tablet |
| | binder; thickening |
| | agent. |
| Propylene paraben | Antimicrobial |
| | preservative |
| Polaxamer 407 or 188 | Dispersing agent; |
| or plain | emulsifying and |
| | coemulsifying agent; |
| | solubilizing agent; tablet |
| | lubricant; wetting |
| | agent. |
| Potassium bicarbonate | Alkalizing agent; |
| | therapeutic agent |
| Potassium sorbate | Antimicrobial |
| | preservative |
| Potato starch | Binder |
| Phosphoric acid | Acidifying agent |
| Polyoxy140 stearate | Emulsifying agent; |
| | solubilizing agent; |
| | wetting agent |
| Sodium starch glycolate | Tablet and capsule |
| | disintegrant |
| Starch pregelatinized | Tablet and capsule |
| | diluent; tablet and |
| | capsule disintegrant; |
| | tablet binder (starch |
| | pregelatinized |
| | Glidant; tablet and |
| | capsule diluent; tablet |
| | and capsule |
| | disintegrant; |
| | tablet binder.(starch, |
| | potato, corn, wheat, |
| | rice) so check the |
| | above and make the |
| | changes |
| Sodium crossmellose | Disintegrant |
| Sodium lauryl sulfate | Anionic surfactant; |
| | detergent; |
| | emulsifying agent; |
| | skin penetrant; tablet and |
| | capsule lubricant; |
| | wetting agent |
| Starch | Glidant; tablet and |
| | capsule diluent; tablet |
| | and capsule |
| | disintegrant; |
| | tablet binder.(starch, |
| | potato, corn, wheat, |
| | rice) combine all the |
| | starches |
| Silicon dioxide | Same as colloidal silicon |
| | dioxide |
| Sodium benzoate | Antimicrobial |
| | preservative; tablet |
| | and capsule lubricant |
| Stearic acid | Emulsifying agent; |
| | solubilizing agent; |
| | tablet and capsule |
| | lubricant. |
| Sucrose | Base for medicated |
| | confectionery; |
| | granulating agent; |
| | sugar coating adjunct; |
| | suspending agent; |
| | sweetening agent; |
| | tablet and capsule |
| | diluent; viscosity- |
| | increasing agent. |
| Sorbic acid | Antimicrobial |
| | preservative |
| Sodium carbonate | Carbonating agent |
| Saccharin sodium | Sweetening agent |
| Sodium alginate | Stabilizing agent; |
| | suspending agent; |
| | tablet and capsule |
| | disintegrant; |
| | tablet binder; viscosity- |
| | increasing agent. |
| Silica gel | Adsorbant |
| Sorbiton monooleate | Solubilizer |
| Sodium stearyl | Tablet and capsule |
| fumarate | lubricant. |
| Sodium chloride | Tablet and capsule |
| | diluent; tonicity agent |
| Sodium metabisulfite | Antioxidant. |
| Sodium citrate | Alkalizing agent; |
| dihydrate | buffering agent; |
| | emulsifier; |
| | sequestering agent. |
| Sodium starch | Binder |
| Sodium carboxy methyl | Coating agent; tablet and |
| cellulose | capsule disintegrant; |
| | tablet binder; stabilizing |
| | agent; suspending |
| | agent; |
| | viscosity-increasing |
| | agent; water- |
| | absorbing agent. |
| Succinic acid | Acidity |
| Sodium propionate | Antimicrobial |
| | preservative |
| Titanium dioxide | Coating agent; opacifier; |
| | pigment |
| Talc | Anticaking agent; |
| | glidant; tablet and |
| | capsule diluent; tablet |
| | and capsule lubricant. |
| Triacetin | Humectant; plasticizer; |
| | solvent |
| Triethyl citrate | Plasticizer |

Also disclosed herein are methods of fabricating the orally dissolvable cannabis tablet 20 or capsule 150. Generally, the methods can include pharmaceutical techniques for mixing and forming tablets or capsules, such as die pressing or encapsulation. In one example, the tablet 20 is made by additive manufacturing. Additive manufacturing involves forming the orally dissolvable cannabis tablet 20 layer-by-layer according to digitized data representing the geometry of the tablet. Three-dimensional printing is one example of additive manufacturing.

FIG. 3 illustrates an example system or machine 30 that is configured to additively form the orally dissolvable cannabis tablet 20. The machine 30 generally includes a dispenser 32, such as a nozzle or head, a drive 34 that is capable of moving the dispenser 32, a controller 36, and one or more source materials 38. As can be appreciated, the machine 30 is shown in a rudimentary form and may also include other features to enhance or facilitate processing.

The controller 36 is configured with software, hardware (e.g., a microprocessor), or a combination thereof, for forming, or “printing,” the orally dissolvable cannabis tablet 20. In this regard, the controller 36 may include or be configured to receive one or more data sets that represent the geometry or a variety of possible selectable geometries of the orally dissolvable cannabis tablet 20. The controller 36 electronically signals the drive 34 to move the dispenser 32 in accordance with the geometry to dispense the orally dissolvable cannabis tablet 20, layer-by-layer.

The one or more source materials include the compositional constituents of the orally dissolvable cannabis tablet 20. These constituents may be pre-mixed and dispensed or may be provided individually or in sub-mixtures that are then mixed and dispensed. The constituents may be mixed in a carrier that facilitates flow and dispensing, such as but not limited to, liquid solvents. In a further example, one or more of the constituents is provided in a discrete cartridge. The one or more cartridges can be used and replaced as needed with a new cartridge.

Treatment or use of the orally dissolvable cannabis tablet 20 can include placement of the orally dissolvable cannabis tablet 20 in the mouth. For example, the orally dissolvable cannabis tablet 20 may be placed under the tongue or between the check and gum. The orally dissolvable cannabis tablet 20 rapidly dissolves upon exposure to saliva in the mouth. The one or more active cannabis-based chemical constituents are then absorbed through the mouth into the bloodstream for near immediate effect. The orally dissolvable cannabis tablet 20 thus provides a new, rapid form for delivering the one or more active chemicals of cannabis into the body, while avoiding the effects associated with inhalation smoking and the long delivery time and inefficient delivery that is associated with ingestion or topical application of cannabis.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. An orally dissolvable cannabis tablet including one or more active cannabis-based chemical constituents and one or more inactive constituents, the active cannabis-based chemical constituents including at least one of cannabinoids or terpenoids.

2. The tablet as recited in claim 1, including 1-100 milligrams of the one or more active cannabis-based chemical constituents and a remainder of the inactive constituents.

3. The tablet as recited in claim 2, wherein the tablet has a total weight of 100-1000 milligrams.

4. The tablet as recited in claim 1, wherein the one or more active cannabis-based chemical constituents are selected from the group consisting of tetrahydrocannabinol, cannabidiol, cannabinol, cannabavarin, cannabigerol, cannabichromene, delta-8-THC, cannabicyclol, cannabitriol, cannabielsoin, myrcene, limonene, and caryophyllene.

5. The tablet as recited in claim 4, wherein the one or more active cannabis-based chemical constituents includes multiple active cannabis-based chemical constituents selected from the group consisting of tetrahydrocannabinol, cannabidiol, cannabinol, cannabavarin, cannabigerol, cannabichromene, delta-8-THC, cannabicyclol, cannabitriol, cannabielsoin, myrcene, limonene, and caryophyllene.

6. The tablet as recited in claim 1, wherein the inactive constituent consists of a predominant amount of a water-soluble solid.

7. The tablet as recited in claim 6, wherein the water-soluble solid is selected from the group consisting of starches, sugars, cellulose solids, and combinations thereof.

8. The tablet as recited in claim 6, wherein the water-soluble solid is starch.

9. The tablet as recited in claim 6, wherein the water-soluble solid is sugar.

10. The tablet as recited in claim 6, wherein the water-soluble solid is cellulose.

11. A machine configured to additively manufacture an orally dissolvable cannabis tablet that includes compositional constituents of one or more active cannabis-based chemical constituents and one or more inactive constituents, the active cannabis-based chemical constituents includes at least one of cannabinoids or terpenoids, the machine including,
one or more cartridges containing one or more of the compositional constituents,
a dispenser connected with the one or more cartridges,
a drive capable of moving the dispenser, and
a controller configured to signal the drive to move the dispenser in accordance with a tablet geometry to dispense the compositional constituents layer-by-layer to form the orally dissolvable cannabis tablet.

12. The machine as recited in claim 11, wherein one or more of the compositional constituents are mixed in a carrier fluid in the one or more cartridges.

13. The machine as recited in claim 11, wherein the controller is configured to receive one or more data sets that represent a variety of possible selectable geometries of the orally dissolvable cannabis tablet.

14. The machine as recited in claim 11, wherein the one or more active cannabis-based chemical constituents are selected from the group consisting of tetrahydrocannabinol, cannabidiol, cannabinol, cannabavarin, cannabigerol, cannabichromene, delta-8-THC, cannabicyclol, cannabitriol, cannabielsoin, myrcene, limonene, and caryophyllene.

15. The machine as recited in claim 11, wherein the one or more active cannabis-based chemical constituents includes multiple active cannabis-based chemical constituents selected from the group consisting of tetrahydrocannabinol, cannabidiol, cannabinol, cannabavarin, cannabigerol, cannabichromene, delta-8-THC, cannabicyclol, cannabitriol, cannabielsoin, myrcene, limonene, and caryophyllene.

16. The machine as recited in claim 15, wherein the inactive constituent consists of a predominant amount of a water-soluble solid.

17. A cannabis tablet or capsule including one or more active cannabis-based chemical constituents and one or more inactive constituents, the active cannabis-based chemical constituents including at least one of cannabinoids or terpenoids.

18. The tablet or capsule as recited in claim 17, including 1-150 milligrams of the one or more active cannabis-based chemical constituents and a remainder of the inactive constituents.

19. The tablet or capsule as recited in claim 17, including 1-250 milligrams of the one or more active cannabis-based chemical constituents and a remainder of the inactive constituents, and the one or more active cannabis-based chemical constituents include tetrahydrocannabinol, cannabidiol, and cannabinol.

20. The tablet or capsule as recited in claim 19, including 1-50 milligrams of the cannabinol.
